(19) United States
(12) Patent Application Publication
(10) Pub. No.: US 2025/0009630 A1
(43) Pub. Date: Jan. 9, 2025

(54) WATER-IN-OIL EMULSION COSMETIC

(71) Applicant: SHISEIDO COMPANY, LTD., Tokyo (JP)

(72) Inventors: Tetsuo EBIHARA, Tokyo (JP); Ikuhiro SUZUKI, Tokyo (JP)

(73) Assignee: SHISEIDO COMPANY, LTD., Tokyo (JP)

(21) Appl. No.: 18/693,158

(22) PCT Filed: Oct. 6, 2022
(86) PCT No.: PCT/JP2022/037382

§ 371 Date: Mar. 19, 2024

(30) Foreign Application Priority Data
Oct. 19, 2021 (JP) 2021-171103

(51) Int. Cl. A61K 8/49; A61K 8/06; A61K 8/19; A61K 8/86; A61K 8/894; A61K 8/92; A61Q 19/00

(52) U.S. Cl. CPC A61K 8/4946; A61K 8/06; A61K 8/19; A61K 8/86; A61K 8/894; A61K 8/92; A61Q 19/00

(57) ABSTRACT

[Problem] To provide a water-in-oil emulsion cosmetic having an excellent feel in use.

[Solution] A water-in-oil emulsion cosmetic including (A) a cyclic carboxamide derivative having a specific structure or its salt, (B) an organo-modified clay mineral, (C) a nonionic surfactant having an HLB of 0.1 to 7.5, (D) an oil, and (E) water.

### TECHNICAL FIELD

The present invention relates to a water-in-oil emulsion cosmetic.

### BACKGROUND ART

In water-in-oil cosmetics, a technique has been proposed in which an organo-modified clay mineral and a specific surfactant are combined for emulsification, and it is known that this technique provides a cosmetic having emulsion stability and good skin conformability (Patent Literature 1). The users of such a cosmetic have desired a more excellent feel in use, and there has been room for further improvement in the feel in use.

Meanwhile, a cyclic carboxamide derivative is known to have an anti-wrinkle effect and a pigmentation inhibitory effect, and is proposed to be blended in a cosmetic and the like (Patent Literature 2).

### CITATION LIST

### Patent Literature

Patent Literature 1: JP 2012-102087 A
Patent Literature 2: WO 2011/040496 A

### SUMMARY OF THE INVENTION

According to the studies of the present inventors, it has been found that use of a cyclic carboxamide derivative in a cosmetic tends to cause a sticky feel. The present inventors have surprisingly found that an excellent feel in use can be achieved by using a water-in-oil cosmetic including a combination of a cyclic carboxamide derivative, an organo-modified clay mineral, and a specific surfactant. The present invention is based on these findings.

According to the present invention, the following invention is provided.

[1] A water-in-oil emulsion cosmetic including:
(A) a cyclic carboxamide derivative or a salt of the cyclic carboxamide derivative, the cyclic carboxamide derivative represented by Formula (1):

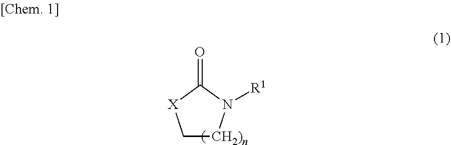

wherein
R¹ represents a hydrocarbon group having 1 to 6 carbon atoms optionally substituted with a hydroxy group or R¹ represents a hydrogen atom,
X represents —CH₂— or —N(R²)— wherein R² represents a hydrocarbon group having 1 to 6 carbon atoms optionally substituted with a hydroxy group or R² represents a hydrogen atom, and
n is an integer of 1 to 3;
(B) an organo-modified clay mineral;
(C) a nonionic surfactant having an HLB of 0.1 to 7.5;
(D) an oil; and
(E) water.
   [2] The cosmetic according to [1],
wherein in Formula (1) of the component (A),
R¹ represents a hydroxyalkyl group having 1 to 3 carbon atoms,
X represents —CH₂— or —NH—, and
n is 1.
   [3] The cosmetic according to [1] or [2], wherein the component (A) is 1-(2-hydroxyethyl)-2-imidazolidinone.
   [4] The cosmetic according to any one of [1] to [3], wherein an amount of the component (A) blended is 0.05 to 5 mass % with respect to a total amount of the cosmetic.
   [5] The cosmetic according to any one of [1] to [4], wherein the component (B) is selected from the group consisting of disteardimonium hectorite, quaternium-18 hectorite, stearalkonium hectorite, and distearyl dimethyl ammonium chloride-treated aluminum magnesium-silicate.
   [6] The cosmetic according to any one of [1] to [5], wherein the component (C) is selected from the group consisting of polyether-modified silicones, polyglycerin-modified silicones, polyglycerin fatty acid esters, and polyoxyalkylene fatty acid esters.
   [7] The cosmetic according to any one of [1] to [6], wherein the component (C) is selected from the group consisting of PEG-9 polydimethylsiloxyethyl dimethicone, PEG-10 dimethicone, lauryl PEG-9 polydimethylsiloxyethyl dimethicone, cetyl PEG/PPG-10/1 dimethicone, bis-butyldimethicone polyglyceryl-3, polyglyceryl-2 diisostearate, polyglyceryl-6 polyricinoleate, and PEG-8 diisostearate.
   [8] The cosmetic according to any one of [1] to [7], wherein (D) the oil is selected from the group consisting of silicone oils, ester oils, hydrocarbon oils, and liquid fatty acids.
   [9] The cosmetic according to any one of [1] to [8], wherein an amount of the component (B) blended is 0.1 to 3 mass % with respect to the total amount of the water-in-oil emulsion cosmetic.
   [10] The cosmetic according to any one of [1] to [9], wherein an amount of the component (C) blended is 0.5 to 5 mass % with respect to the total amount of the cosmetic.
   [11] The cosmetic according to any one of [1] to [10], wherein an amount of the component (D) blended is 10 to 70 mass % with respect to the total amount of the cosmetic.

According to the present invention, a water-in-oil emulsion cosmetic having an excellent feel in use can be provided. In particular, a mellow feel is imparted during application, a sticky feel is suppressed after application, and a moist feel can be imparted to the skin.

### BEST MODE FOR CARRYING OUT THE INVENTION

The present invention relates to a water-in-oil emulsion cosmetic (hereinafter, sometimes referred to as a cosmetic) including (A) a cyclic carboxamide derivative having a specific structure or its salt, (B) an organo-modified clay mineral, (C) a nonionic surfactant having an HLB of 0.1 to 7.5, (D) an oil, and (E) water.

### (A) Cyclic Carboxamide Derivative or its Salt

The cosmetic according to the present invention includes a cyclic carboxamide derivative represented by Formula (1) or its salt (hereinafter, sometimes referred to as a component (A), and the same applies to other components).

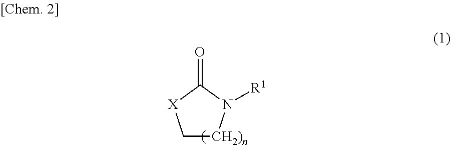

In Formula (1),
R¹ represents a hydrocarbon group having 1 to 6 carbon atoms optionally substituted with a hydroxy group or R¹ represents a hydrogen atom,
X represents —CH₂— or —N(R²)— wherein R² represents a hydrocarbon group having 1 to 6 carbon atoms optionally substituted with a hydroxy group or R² represents a hydrogen atom, and
n is an integer of 1 to 3

The hydrocarbon group is not particularly limited, and may be, for example, an alkyl group, a cycloalkyl group, an alkenyl group, an alkynyl group, a cycloalkylalkyl group, a haloalkyl group, an alkoxyalkyl group, or an alkoxycarbonylalkyl group, and is preferably an alkyl group.

In a preferred embodiment, in Formula (1) of the component (A),
R¹ represents a hydroxyalkyl group having 1 to 3 carbon atoms,
X represents —CH₂— or —NH—, and
n is 1.

Specific examples of the cyclic carboxamide derivative represented by Formula (1) include the following.

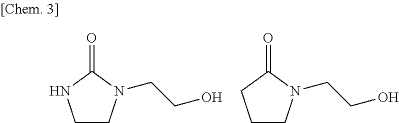

The component (A) is most preferably 1-(2-hydroxyethyl)-2-imidazolidinone.

The component (A) may be a salt of the cyclic carboxamide derivative represented by Formula (1). The kind of the salt is not particularly limited as long as the salt is pharmacologically acceptable, and the salt may be an inorganic salt or an organic salt. Examples of the inorganic salt include a hydrochloride, a sulfate, a phosphate, a hydrobromide, a sodium salt, a potassium salt, a magnesium salt, a calcium salt, a magnesium salt, and an ammonium salt. Examples of the organic salt include an acetate, a lactate, a maleate, a fumarate, a tartrate, a methanesulfonate, a p-toluenesulfonate, a triethanolamine salt, and an amino acid salt.

One or more kinds of the component (A) can be blended. The amount of the component (A) blended is preferably 0.05 to 5 mass %, more preferably 0.08 to 3.5 mass %, and still more preferably 0.5 to 2.0 mass % with respect to the total amount of the cosmetic.

### (B) Organo-Modified Clay Mineral

The cosmetic according to the present invention includes (B) an organo-modified clay mineral. The component (B) is used as an emulsification aid. The component (B) is, for example, one kind of colloidal hydrous aluminum silicate having a three-layer structure, and examples of the component (B) include those obtained by modifying a clay mineral represented by the following formula with a quaternary ammonium salt cationic surfactant.

(X,Y)₂₋₃(Si,Al)₄O₁₀(OH)₂Z_{1/3}·nH₂O

In the formula,
X is Al, Fe(III), Mn(III), or Cr(III),
Y is Mg, Fe(II), Ni, Zn, or Li, and
Z is K, Na, or Ca.

The quaternary ammonium salt cationic surfactant used here is represented as follows.

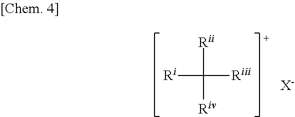

In the formula,
Rⁱ represents an alkyl group having 10 to 22 carbon atoms or a benzyl group,
Rⁱⁱ represents a methyl group or an alkyl group having 10 to 22 carbon atoms,
Rⁱⁱⁱ and R^{iv} each independently represent an alkyl group or a hydroxyalkyl group having 1 to 3 carbon atoms, and
X is a halogen atom or a methyl sulfate residue.

Examples of such a quaternary ammonium salt cationic surfactant include dodecyltrimethylammonium chloride, myristyltrimethylammonium chloride, cetyltrimethylammonium chloride, stearyltrimethylammonium chloride, arachyltrimethylammonium chloride, behenyl trimethyl ammonium chloride, myristyldimethylethylammonium chloride, cetyldimethylethylammonium chloride, stearyldimethylethylammonium chloride, arachyldimethylethylammonium chloride, behenyldimethylethylammonium chloride, myristyldiethylmethylammonium chloride, cetyldiethylmethylammonium chloride, stearyldiethylmethylammonium chloride, arachyldiethylmethylammonium chloride, behenyldiethylmethylammonium chloride, benzyldimethylmyristylammonium chloride, benzyldimethylcetylammonium chloride, benzyldimethylstearylammonium chloride, benzyldimethylbehenylammonium chloride, benzylmethylethylcetylammonium chloride, benzylmethylethylstearylammonium chloride, dibehenyldihydroxyethylammonium chloride, corresponding bromides, and dipalmitylpropylethylammonium methylsulfate. In practice of the present invention, one or more kinds thereof are optionally selected.

The component (B) is preferably selected from the group consisting of disteardimonium hectorite, quaternium-18 hectorite, stearalkonium hectorite, and distearyl dimethyl ammonium chloride-treated aluminum magnesium-silicate. Examples of the commercially available product include BENTONE 27 (benzyldimethylstearylammonium chloride-treated hectorite by Elementis) and BENTONE 38 (distearyldimethylammonium chloride-treated hectorite by Elementis). The component (B) in a powder state can be used, in addition to the component (B) in a state of being dispersed in a dispersion medium.

One or more kinds of the component (B) can be blended. The amount of the component (B) blended is preferably 0.1 to 3 mass %, and more preferably 0.5 to 2 mass % with respect to the total amount of the cosmetic.

### (C) Nonionic Surfactant Having HLB of 0.1 to 7.5

The cosmetic according to the present invention includes (C) a nonionic surfactant having an HLB of 0.1 to 7.5. The component (C) preferably has an HLB of 0.5 to 7.0. Here, in the present invention, the hydrophile-lipophile balance (HLB) of the component (C) can be determined with the Griffin method.

The component (C) is preferably selected from the group consisting of polyether-modified silicones, polyglycerin-modified silicones, polyglycerin fatty acid esters, and polyoxyalkylene fatty acid esters.

Examples of the polyether-modified silicones include PEG-9 polydimethylsiloxyethyl dimethicone, PEG-10 dimethicone, lauryl PEG-9 polydimethylsiloxyethyl dimethicone, and cetyl PEG/PPG-10/1 dimethicone.

Examples of the polyglycerin-modified silicones include bis-butyldimethicone polyglyceryl-3.

Examples of the polyglycerin fatty acid esters include polyglyceryl-2 diisostearate, polyglyceryl-6 triisostearate, polyglyceryl-10 pentastearate, polyglyceryl-10 pentaisostearate, polyglyceryl-10 pentaoleate, and polyglyceryl-6 polyricinoleate.

Examples of the polyoxyalkylene fatty acid esters include PEG-8 diisostearate, PEG-4 sorbitan triisostearate, and PEG-6 sorbitol hexastearate.

The component (C) is more preferably selected from the group consisting of PEG-9 polydimethylsiloxyethyl dimethicone, PEG-10 dimethicone, lauryl PEG-9 polydimethylsiloxyethyl dimethicone, cetyl PEG/PPG-10/1 dimethicone, bis-butyldimethicone polyglyceryl-3, polyglyceryl-2 diisostearate, polyglyceryl-6 polyricinoleate, and PEG-8 diisostearate.

One or more kinds of the component (C) can be blended. The amount of the component (C) blended is preferably 0.5 to 5 mass %, and more preferably 1 to 3 mass % with respect to the total amount of the cosmetic.

### (D) Oil

The cosmetic according to the present invention includes (D) an oil. Examples of the component (D) include silicone oils, hydrocarbon oils, higher fatty acids, higher alcohols, ester oils, liquid oils, solid oils, and semi-solid oils. The component (D) is preferably selected from the group consisting of silicone oils, ester oils, and hydrocarbon oils.

Examples of the silicone oils include chain polysiloxanes (such as dimethicone, diphenylsiloxy phenyl trimethicone, and diphenylpolysiloxane), cyclic polysiloxanes (such as octamethylcyclotetrasiloxane, decamethylcyclopentasiloxane, and dodecamethylcyclohexasiloxane), silicone resins forming a three-dimensional network structure, silicone rubber, various modified polysiloxanes (such as an amino-modified polysiloxane, a polyether-modified polysiloxane, an alkyl-modified polysiloxane, and a fluorine-modified polysiloxane), and acrylic silicones, and chain polysiloxanes are preferable.

Examples of the ester oils include octyl octanoate, nonyl nonanoate, cetyl octanoate, isopropyl myristate, octyldodecyl myristate, isopropyl palmitate, ethylhexyl palmitate, butyl stearate, hexyl laurate, myristyl myristate, decyl oleate, hexyldecyl dimethyl octanoate, cetyl lactate, myristyl lactate, lanolin acetate, isocetyl stearate, isocetyl isostearate, cholesteryl 12-hydroxystearate, ethylene glycol di-2-ethylhexanoate, dipentaerythritol fatty acid ester, N-alkylglycol monoisostearate, neopentyl glycol dicaprate, tripropylene glycol pivalate, PPG-3 dipivalate, diisostearyl malate, glyceryl di-2-heptylundecanoate, glyceryl diisostearate, trimethylolpropane tri-2-ethylhexanoate, trimethylolpropane triisostearate, pentaerythritol tetra-2-ethylhexanoate, glyceryl tri-2-ethylhexanoate (triethylhexanoin), glyceryl trioctanoate, glyceryl triisopalmitate, trimethylolpropane triisostearate, cetyl 2-ethylhexanoate-2-ethylhexyl palmitate, glycerin trimyristate, glyceride tri-2-heptylundecanoate, castor oil fatty acid methyl ester, oleyl oleate, acetoglyceride, 2-heptylundecyl palmitate, diisobutyl adipate, phytosteryl/octyldodecyl lauroyl glutamate, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, triethyl citrate, cetyl ethylhexanoate, and phytosteryl macadamiate.

Examples of the hydrocarbon oils include isododecane, isohexadecane, isoparaffin, mineral oil (liquid paraffin), ozokerite, squalane, pristane, paraffin, ceresin, squalene, petrolatum, microcrystalline wax, and hydrogenated polydecene.

Examples of the higher fatty acids include lauric acid, myristic acid, palmitic acid, stearic acid, behenic acid, oleic acid, undecylenic acid, tall oil acid, isostearic acid, linolic acid, linoleic acid, eicosapentaenoic acid (EPA), and docosahexaenoic acid (DHA).

Examples of the higher alcohols include straight-chain alcohols (such as lauryl alcohol, cetyl alcohol, stearyl alcohol, behenyl alcohol, myristyl alcohol, oleyl alcohol, and cetostearyl alcohol) and branched alcohols (such as lanolin alcohol, cholesterol, phytosterol, hexyldodecanol, isostearyl alcohol, and octyldodecanol).

Examples of the liquid oils include avocado oil, camellia oil, macadamia nut oil, corn oil, olive oil, rapeseed oil, sesame oil, persic oil, wheat germ oil, sasanqua oil, castor oil, linseed oil, safflower oil, cottonseed oil, perilla oil, soybean oil, peanut oil, tea seed oil, kaya oil, rice bran oil, paulownia oil, Japanese tung oil, jojoba oil, germ oil, and triglycerin oil. Examples of the solid oils include cacao butter, coconut oil, hardened coconut oil, palm oil, palm kernel oil, hydrogenated palm oil, Japan wax kernel oil, hardened oils, Japan wax, and hardened castor oil. Examples of the semi-solid oils include shea butter, partially hydrogenated coconut oil, and partially hydrogenated jojoba oil.

One or more kinds of the component (D) can be blended. The amount of the component (D) blended is preferably 10 to 70 mass %, more preferably 15 to 60 mass %, and still more preferably 20 to 50 mass % with respect to the total amount of the cosmetic.

### (E) Water

The cosmetic according to the present invention includes (E) water. As the water, water used in cosmetics, quasi-drugs, and the like can be used, and for example, purified water, ion-exchanged water, tap water, or the like can be used.

The amount of the water blended is preferably 20 to 80 mass %, and more preferably 30 to 70 mass % with respect to the total amount of the cosmetic according to the present invention.

In the cosmetic in the present invention, an optional component usually used in cosmetics and pharmaceuticals can be blended in addition to the above-described components. Examples of the optional component include the following components, and one or more kinds of them can be blended as long as an effect of the present invention is exhibited.

Examples of the moisturizer include polyethylene glycol, propylene glycol, dipropylene glycol (DPG), glycerin, 1,3-butylene glycol (BG), erythritol, xylitol, sorbitol, maltitol, chondroitin sulfate, hyaluronic acid, mucoitinsulfuric acid, charonin acid, atelocollagen, cholesteryl-12-hydroxystearate, sodium lactate, bile acid salts, dl-pyrrolidone carboxylates, short-chain soluble collagen, a chestnut rose extract, a yarrow extract, and a melilot extract.

Examples of the lower alcohol include ethanol, 1-propanol, 2-propanol, isobutyl alcohol, and t-butyl alcohol.

Examples of the thickener include gum arabic, carrageenan, karaya gum, tragacanth gum, carob gum, quince seed (Pyrus cydonia), casein, dextrin, gelatin, sodium pectinate, sodium alginate, methylcellulose, ethylcellulose, CMC, hydroxyethylcellulose, hydroxypropylcellulose, PVA, PVM, PVP, sodium polyacrylate, a carboxyvinyl polymer, locust bean gum, guar gum, tamarind gum, dialkyldimethylammonium sulfate cellulose, xanthan gum, magnesium aluminum silicate, bentonite, hectorite, magnesium aluminum silicate (VEEGUM), laponite, and silicic anhydride.

Examples of the sequestering agent include 1-hydroxyethane-1,1-diphosphonic acid, 1-hydroxyethane-1,1-diphosphonic acid tetrasodium salt, edetate disodium, edetate trisodium, edetate tetrasodium, sodium citrate, sodium polyphosphate, sodium metaphosphate, gluconic acid, phosphoric acid, citric acid, succinic acid, edetic acid, and trisodium hydroxyethyl ethylenediamine triacetate.

Examples of the neutralizer include 2-amino-2-methyl-1-propanol, 2-amino-2-methyl-1,3-propanediol, potassium hydroxide, sodium hydroxide, triethanolamine, and sodium carbonate.

Examples of a pH adjuster include buffers such as lactic acid-sodium lactate, citric acid-sodium citrate, and succinic acid-sodium succinate.

Examples of the antioxidant include dibutylhydroxytoluene, butylhydroxyanisole, sodium pyrosulfite, and gallic acid esters.

Examples of the preservative include paraoxybenzoic acid esters such as methylparaben, ethylparaben, and butylparaben, benzoic acid, salicylic acid, sorbic acid, parachlorometacresol, hexachlorophene, benzalkonium chloride, chlorhexidine chloride, trichlorocarbanilide, photosensitizers, phenoxyethanol, and chlorphenesin.

Examples of the drug include ascorbic acid (vitamin C), tranexamic acid, kojic acid, ellagic acid, arbutin, alkoxysalicylic acid, glycyrrhizic acid, tocopherol, retinol, salts and derivatives thereof (for example, sodium L-ascorbate, L-ascorbic acid ester magnesium salt, L-ascorbic acid glucoside, 2-O-ethyl-L-ascorbic acid, 3-O-ethyl-L-ascorbic acid, 4-methoxysalicylic acid sodium salt, 4-methoxysalicylic acid potassium salt, dipotassium glycyrrhizinate, stearyl glycyrrhizinate, tocopherol acetate, retinol acetate, and retinol palmitate), caffeine, tannin, verapamil and its derivatives, a licorice extract, glabridin, a hot water extract of Pyracantha fortuneana seed, various herbal medicines, hydrolyzed silk, hydrolyzed conchiolin, a tea extract, a Potentilla erecta root extract, an Angelica keiskei leaf/stem extract, an Aloe barbadensis leaf extract, a cherry leaf extract, an Angelica acutiloba Kitagawa root extract, a Citrus depressa peel extract, an Iris florentina root extract, an Eucheuma serra/Grateloupia sparsa/Saccharina angustata/Ulva linza/Undaria pinnatifida extract, a Typha angustifolia spike extract, an Isodonis japonicus leaf/stalk extract, a Camellia japonica seed extract, a Saccharina angustata/Undaria pinnatifida extract, a Bupleurum falcatum root extract, a Nasturtium officinale leaf/stem extract, a Cinnamomum cassia bark extract, a rosemary leaf oil, a lavender oil, glutamic acid, trimethylglycine, chlorphenesin, and menthoxypropanediol.

In addition, an ultraviolet absorber, a powder component, an aroma chemical, and the like can also be appropriately blended.

Examples of the cosmetic according to the present invention include skin care cosmetics (such as lotions, milky lotions, creams, beauty essences, packs, and masks), makeup cosmetics (such as foundations and makeup bases), skin cleaning agents (such as face washes and makeup removers), sunscreen cosmetics, and ointments. These embodiments are merely examples, and the cosmetic according to the present invention is not limited to these embodiments.

The method of emulsification for the cosmetic of the present invention is not particularly limited, and the cosmetic can be produced in accordance with a conventional method.

The hardness is not particularly limited, but is preferably 5 to 25 N, and more preferably 8 to 18 N. The hardness can be measured at 25° C. using a rheometer manufactured by RHEOTECH (pressure sensing shaft 11.3ϕ, needle penetration speed 10 cm/min, needle penetration depth 10 mm).

### EXAMPLES

The present invention will be described specifically with reference to the following Examples, but the present invention is not limited to these Examples. The content is shown in mass % with respect to the total amount unless otherwise specified.

### Examples 1 to 16 and Comparative Examples 1 to 4

Cosmetics of Examples 1 to 16 and Comparative Examples 1 to 4 were prepared at combinations shown in Tables 1 and 2. The numerical values of the components in the tables are shown in mass %. The unit of hardness is N.

TABLE 1

| | | | Example | | | | | | | | | |
|---|---|---|---|---|---|---|---|---|---|---|---|---|
| | | HLB | 1 | 2 | 3 | 4 | 5 | 6 | 7 | 8 | 9 | 10 |
| (A) | 1-(2-Hydroxyethyl)-2-imidazolidinone | | 1.5 | 1.5 | 1.5 | 1.5 | 0.1 | 1.5 | 1.5 | 1.5 | 1.5 | 1.5 |
| (B) | Disteardimonium hectorite | | 0.8 | — | — | — | 0.8 | 0.8 | 0.8 | 0.8 | 0.8 | 0.8 |
| | Quaternium-18 hectorite | | — | 0.8 | — | — | — | — | — | — | — | — |
| | Stearalkonium hectorite | | — | — | 0.8 | — | — | — | — | — | — | — |
| | Magnesium aluminum silicate | | — | — | — | 0.6 | — | — | — | — | — | — |
| | Distearyldimonium chloride | | — | — | — | 0.2 | — | — | — | — | — | — |
| (C) | PEG-9 polydimethylsiloxyethyl | 4 | — | — | — | — | — | 2 | — | — | — | — |
| | dimethicone | | | | | | | | | | | |
| | PEG-10 dimethicone | 4.5 | 2 | 2 | 2 | 2 | 2 | — | — | — | — | — |
| | PEG-10 dimethicone | 2 | — | — | — | — | — | — | 2 | — | — | — |
| | Lauryl PEG-9 polydimethylsiloxyethyl | 3 | — | — | — | — | — | — | — | 2 | — | — |
| | dimethicone | | | | | | | | | | | |
| | Bis-butyldimethicone polyglyceryl-3 | 0.5 | — | — | — | — | — | — | — | — | 2 | — |
| | Cetyl PEG/PPG-10/1 dimethicone | 5 | — | — | — | — | — | — | — | — | — | 2 |
| | Cetyl PEG/PPG-10/1 dimethicone | 5 | — | — | — | — | — | — | — | — | — | — |
| | Polyglyceryl-2 diisostearate | 3.6 | — | — | — | — | — | — | — | — | — | — |
| | PEG-8 diisostearate | 7 | — | — | — | — | — | — | — | — | — | — |
| | Polyglyceryl-6 polyricinoleate | 3.3 | — | — | — | — | — | — | — | — | — | — |
| (D) | Hydrogenated polyisobutene | | — | — | — | — | — | — | — | — | — | — |
| | Hydrogenated polydecene | | 8 | 8 | 8 | 8 | — | — | — | — | — | — |
| | Isododecane | | — | — | — | — | — | 4 | — | — | — | — |
| | Dimethicone (kinematic viscosity 1.5 mm²/s) | | — | — | — | — | — | 4 | — | — | — | — |
| | Dimethicone (kinematic viscosity 6.0 mm²/s) | | 3 | 3 | 3 | 3 | 12 | 3 | 12 | 12 | 12 | 12 |
| | Diphenylsiloxy phenyl trimethicone | | 3 | 3 | 3 | 3 | 8 | 3 | 8 | 8 | 8 | 8 |
| | Caprylyl methicone | | — | — | — | — | 6 | — | 6 | 6 | 6 | 6 |
| | Triethylhexanoin | | 12 | 12 | 12 | 12 | 3 | 12 | 3 | 3 | 3 | 3 |
| | Cetyl ethylhexanoate | | 6 | 6 | 6 | 6 | 3 | 6 | 3 | 3 | 3 | 3 |
| | PPG-3 dipivalate | | — | — | — | — | — | — | — | — | — | — |
| | Petrolatum | | — | — | — | — | — | — | — | — | — | — |
| | Squalane | | — | — | — | — | — | — | — | — | — | — |
| | Hydrogenated palm oil | | — | — | — | — | — | — | — | — | — | — |
| | Palm kernel oil | | — | — | — | — | — | — | — | — | — | — |
| | Palm oil | | — | — | — | — | — | — | — | — | — | — |
| | Phytosteryl/octyldodecyl lauroyl glutamate | | — | — | — | — | — | — | — | — | — | — |
| | Myristyl myristate | | — | — | — | — | — | — | — | — | — | — |
| (E) | Water | | Bal- | Bal- | Bal- | Bal- | Bal- | Bal- | Bal- | Bal- | Bal- | Bal- |

| | | | | | | | | | | |
|---|---|---|---|---|---|---|---|---|---|---|
| | ance | ance | ance | ance | ance | ance | ance | ance | ance | ance |
| Ethanol | 3 | 3 | 3 | 3 | 3 | 3 | 3 | 3 | 3 | 3 |
| Glycerin | 3 | 3 | 3 | 3 | 3 | 3 | 3 | 3 | 3 | 3 |
| DPG | 2 | 2 | 2 | 2 | 2 | 2 | 2 | 2 | 2 | 2 |
| BG | 2 | 2 | 2 | 2 | 2 | 2 | 2 | 2 | 2 | 2 |
| Citric acid | 0.2 | 0.2 | 0.2 | 0.2 | 0.2 | 0.2 | 0.2 | 0.2 | 0.2 | 0.2 |
| Sodium citrate | 0.8 | 0.8 | 0.8 | 0.8 | 0.8 | 0.8 | 0.8 | 0.8 | 0.8 | 0.8 |
| Phenoxyethanol | 0.5 | 0.5 | 0.5 | 0.5 | 0.5 | 0.5 | 0.5 | 0.5 | 0.5 | 0.5 |
| Chlorphenesin | 0.2 | 0.2 | 0.2 | 0.2 | 0.2 | 0.2 | 0.2 | 0.2 | 0.2 | 0.2 |
| Total | 100 | 100 | 100 | 100 | 100 | 100 | 100 | 100 | 100 | 100 |
| Absence of sticky feel | A | A | A | A | A | A | A | A | A | A |
| Mellow feel | A | A | A | A | C | A | A | A | A | A |
| Moist feel | A | A | A | A | B | A | A | A | A | A |
| Emulsion stability | ◯ | ◯ | ◯ | ◯ | ◯ | ◯ | ◯ | ◯ | ◯ | ◯ |
| Hardness (ϕ11.3, 10 mm needle penetration) | 13 | 12 | 13 | 13 | 11 | 13 | 14 | 14 | 13 | 11 |

TABLE 2

| | | | Example | | | | | | Comparative Example | | | |
|---|---|---|---|---|---|---|---|---|---|---|---|---|
| | | HLB | 11 | 12 | 13 | 14 | 15 | 16 | 1 | 2 | 3 | 4 |
| (A) | 1-(2-hydroxyethyl)-2-imidazolidinone | | 1.5 | 1.5 | 1.5 | 1.5 | 1.5 | 1.5 | — | 1.5 | 1.5 | 1.5 |
| (B) | Disteardimonium hectorite | | 0.8 | 0.8 | 0.8 | 0.8 | 0.8 | 0.8 | 0.8 | — | 0.8 | — |
| | Quaternium-18 hectorite | | — | — | — | — | — | — | — | — | — | — |
| | Stearalkonium hectorite | | — | — | — | — | — | — | — | — | — | — |
| | Magnesium aluminum silicate | | — | — | — | — | — | — | — | — | — | — |
| | Distearyldimonium chloride | | — | — | — | — | — | — | — | — | — | — |
| PEG/PPG-19/19 dimethicone | | | — | — | — | — | — | — | — | — | — | 0.7 |
| (C) | PEG-9 polydimethylsiloxyethyl | 4 | — | — | — | — | — | — | — | — | — | |
| | dimethicone | | | | | | | | | | | |
| | PEG-10 dimethicone | 4.5 | — | — | — | — | 2 | 2 | 2 | 2 | — | 2 |
| | PEG-10 dimethicone | 2 | — | — | — | — | — | — | — | — | — | — |
| | Lauryl PEG-9 polydimethylsiloxyethyl | 3 | — | — | — | — | — | — | — | — | — | — |
| | dimethicone | | | | | | | | | | | |
| | Bis-butyldimethicone polyglyceryl-3 | 0.5 | — | — | — | — | — | — | — | — | — | — |
| | Cetyl PEG/PPG-10/1 dimethicone | 5 | — | — | — | — | — | — | — | — | — | — |
| | Cetyl PEG/PPG-10/1 dimethicone | 5 | 2 | — | — | — | — | — | — | — | — | — |
| | Polyglyceryl-2 diisostearate | 3.6 | — | 2 | — | — | — | — | — | — | — | — |
| | PEG-8 diisostearate | 7 | — | — | 2 | — | — | — | — | — | — | — |
| | Polyglyceryl-6 polyricinoleate | 3.3 | — | — | — | 2 | — | — | — | — | — | — |
| (D) | Hydrogenated polyisobutene | | — | — | — | — | — | — | — | — | — | 0.7 |
| | Hydrogenated polydecene | | 8 | 8 | 8 | 8 | — | — | 8 | 8 | 8 | 8 |
| | Isododecane | | — | — | — | — | — | — | — | — | — | — |
| | Dimethicone (kinematic viscosity 1.5 mm²/s) | | — | — | — | — | — | — | — | — | — | — |
| | Dimethicone (kinematic viscosity 6.0 mm²/s) | | 3 | 3 | 3 | 3 | 12 | 12 | 3 | 3 | 3 | 3 |
| | Diphenylsiloxy phenyl trimethicone | | 3 | 3 | 3 | 3 | 8 | 8 | 3 | 3 | 3 | 3 |
| | Caprylyl methicone | | — | — | — | — | — | — | — | — | — | — |
| | Triethylhexanoin | | 12 | 12 | 12 | 12 | — | — | 12 | 12 | 12 | 12 |
| | Cetyl ethylhexanoate | | 6 | 6 | 6 | 6 | — | — | 6 | 6 | 6 | 6 |
| | PPG-3 dipivalate | | — | — | — | — | — | 4 | — | — | — | — |
| | Petrolatum | | — | — | — | — | — | 4 | — | — | — | — |
| | Squalane | | — | — | — | — | — | 4 | — | — | — | — |
| | Hydrogenated palm oil | | — | — | — | — | 2 | — | — | — | — | — |
| | Palm kernel oil | | — | — | — | — | 1.2 | — | — | — | — | — |
| | Palm oil | | — | — | — | — | 0.8 | — | — | — | — | — |
| | Phytosteryl/octyldodecyl lauroyl glutamate | | — | — | — | — | 4 | — | — | — | — | — |
| | Myristyl myristate | | — | — | — | — | 4 | — | — | — | — | — |
| (E) | Water | | Bal- | Bal- | Bal- | Bal- | Bal- | Bal- | Bal- | Bal- | Bal- | Bal- |

| | | | | | | | | | | |
|---|---|---|---|---|---|---|---|---|---|---|
| | ance | ance | ance | ance | ance | ance | ance | ance | ance | ance |
| Ethanol | 3 | 3 | 3 | 3 | 3 | 3 | 3 | 3 | 3 | 3 |
| Glycerin | 3 | 3 | 3 | 3 | 3 | 3 | 3 | 3 | 3 | 3 |
| DPG | 2 | 2 | 2 | 2 | 2 | 2 | 2 | 2 | 2 | 2 |
| BG | 2 | 2 | 2 | 2 | 2 | 2 | 2 | 2 | 2 | 2 |
| Citric acid | 0.2 | 0.2 | 0.2 | 0.2 | 0.2 | 0.2 | 0.2 | 0.2 | 0.2 | 0.2 |
| Sodium citrate | 0.8 | 0.8 | 0.8 | 0.8 | 0.8 | 0.8 | 0.8 | 0.8 | 0.8 | 0.8 |
| Phenoxyethanol | 0.5 | 0.5 | 0.5 | 0.5 | 0.5 | 0.5 | 0.5 | 0.5 | 0.5 | 0.5 |
| Chlorphenesin | 0.2 | 0.2 | 0.2 | 0.2 | 0.2 | 0.2 | 0.2 | 0.2 | 0.2 | 0.2 |
| Total | 100 | 100 | 100 | 100 | 100 | 100 | 100 | 100 | 100 | 100 |
| Absence of sticky feel | A | A | A | A | A | A | A | A | A | D |
| Mellow feel | A | A | A | A | A | A | D | D | D | D |
| Moist feel | A | A | A | A | A | A | C | C | C | C |
| Emulsion stability | ◯ | ◯ | ◯ | ◯ | ◯ | ◯ | ◯ | X | X | ◯ |
| Hardness (ϕ11.3, 10 mm needle penetration) | 12 | 13 | 12 | 13 | 10 | 11 | 14 | — | — | 15 |

The cosmetic prepared above was applied to the skin of 10 expert panelists, and the panelists evaluated “absence of a sticky feel” after the application, a “mellow feel” during the application, and a “moist feel” after the application. Determination was made in accordance with the following criteria on the basis of the evaluation of each expert panelist. The obtained results are as shown in Tables 1 and 2.

### [Absence of Sticky Feel]

A: Eight or more of the 10 panelists answered that a sticky feel was absent.

B: Five or more and 7 or less of the 10 panelists answered that a sticky feel was absent.

C: Two or more and 4 or less of the 10 panelists answered that a sticky feel was absent.

D: One or less of the 10 panelists answered that a sticky feel was absent.

### [Mellow Feel]

A: Eight or more of the 10 panelists answered that a mellow feel was present.

B: Five or more and 7 or less of the 10 panelists answered that a mellow feel was present.

C: Two or more and 4 or less of the 10 panelists answered that a mellow feel was present.

D: One or less of the 10 panelists answered that a mellow feel was present.

### [Moist Feel]

A: Eight or more of the 10 panelists answered that a moist feel was present.

B: Five or more and 7 or less of the 10 panelists answered that a moist feel was present.

C: Two or more and 4 or less of the 10 panelists answered that a moist feel was present.

D: One or less of the 10 panelists answered that a moist feel was present.

### [Emulsion Stability]

A container made of a resin containing polyethylene terephthalate as a main component and a container made of glass were filled with each cosmetic prepared above and left to stand at 40° C. for one month, and then whether peeling or slippage from the wall occurred between the container and the content was examined with naked eyes, and the emulsion stability was determined in accordance with the following criteria.
∘: No peeling or slippage from the wall is observed between the container and the content.
x: Peeling or slippage from the wall is observed between the container and the content.

### [Hardness]

The hardness of each cosmetic prepared above was measured at 25° C. using a rheometer manufactured by RHEOTECH (pressure sensing shaft 11.3 ϕ, needle penetration speed 10 cm/min, needle penetration depth 10 mm). The obtained results are as shown in Tables 1 and 2.

1. A water-in-oil emulsion cosmetic comprising:
(A) a cyclic carboxamide derivative or a salt of the cyclic carboxamide derivative, the cyclic carboxamide derivative represented by Formula (1): 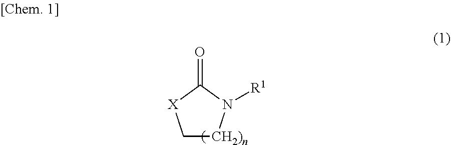
wherein
R¹ represents a hydrocarbon group having 1 to 6 carbon atoms optionally substituted with a hydroxy group or R¹ represents a hydrogen atom,
X represents —CH₂— or —N(R²)— wherein R² represents a hydrocarbon group having 1 to 6 carbon atoms optionally substituted with a hydroxy group or R² represents a hydrogen atom, and
n is an integer of 1 to 3;
(B) an organo-modified clay mineral;
(C) a nonionic surfactant having an HLB of 0.1 to 7.5;
(D) an oil; and
(E) water.

2. The cosmetic according to claim 1,
wherein in Formula (1) of the component (A),
R¹ represents a hydroxyalkyl group having 1 to 3 carbon atoms,
X represents —CH₂— or —NH—, and
n is 1.

3. The cosmetic according to claim 1, wherein the component (A) is 1-(2-hydroxyethyl)-2-imidazolidinone.

4. The cosmetic according to claim 1, wherein an amount of the component (A) blended is 0.05 to 5 mass % with respect to a total amount of the cosmetic.

5. The cosmetic according to claim 1, wherein the component (B) is selected from the group consisting of disteardimonium hectorite, quaternium-18 hectorite, stearalkonium hectorite, and distearyl dimethyl ammonium chloride-treated aluminum magnesium-silicate.

6. The cosmetic according to claim 1, wherein the component (C) is selected from the group consisting of polyether-modified silicones, polyglycerin-modified silicones, polyglycerin fatty acid esters, and polyoxyalkylene fatty acid esters.

7. The cosmetic according to claim 1, wherein the component (C) is selected from the group consisting of PEG-9 polydimethylsiloxyethyl dimethicone, PEG-10 dimethicone, lauryl PEG-9 polydimethylsiloxyethyl dimethicone, cetyl PEG/PPG-10/1 dimethicone, bis-butyldimethicone polyglyceryl-3, polyglyceryl-2 diisostearate, polyglyceryl-6 polyricinoleate, and PEG-8 diisostearate.

8. The cosmetic according to claim 1, wherein (D) the oil is selected from the group consisting of silicone oils, ester oils, and hydrocarbon oils.

9. The cosmetic according to claim 1, wherein an amount of the component (B) blended is 0.1 to 3 mass % with respect to the total amount of the cosmetic.

10. The water-in-oil emulsion cosmetic according to claim 1, wherein an amount of the component (C) blended is 0.5 to 5 mass % with respect to the total amount of the cosmetic.

11. The cosmetic according to claim 1, wherein an amount of the component (D) blended is 10 to 70 mass % with respect to the total amount of the cosmetic.
